(19) United States
(12) Patent Application Publication
(10) Pub. No.: US 2008/0119941 A1
(43) Pub. Date: May 22, 2008

(54) TIBIA IMPACTER

(76) Inventors: Jai-Gon Seo, Seoul (KR); Chong-Bum Kim, Seoul (KR)

Correspondence Address: PARK LAW FIRM, 3255 WILSHIRE BLVD, SUITE 1110, LOS ANGELES, CA, 90010, US

(21) Appl. No.: 11/942,146

(22) Filed: Nov. 19, 2007

(30) Foreign Application Priority Data
Nov. 22, 2006 (KR) 10-2006-115918

(51) Int. Cl. A61F 2/38

(52) U.S. Cl. 623/20.34; 623/20.32

(57) ABSTRACT

The present invention discloses a tibia impacter provided with an alignment guide function that helps an artificial tibia implant to be adjusted to the level condition when bone cement is applied to the top surface of the cut tibia and an artificial tibia implant is driven in and fixed. The tibia impacter comprises a guide bar which is installed vertically on one side of the tibia impacter for comparison with the mechanical axis of the tibia to compare the mechanical axis of the tibia and the guide bar, so that the level condition of the artificial tibia implant mounted on the top surface of tibia can be confirmed.

This application claims priority to Korean Patent Application No. 2006-00000, filed on Nov. 0, 2006, in the Korean Intellectual Property Office, the entire contents of which are hereby incorporated by reference.

### BACKGROUND OF THE INVENTION

1. Field of the Invention

The present invention relates to a tibia impacter, and more specifically to a tibia impacter provided with an alignment guide function that helps an artificial tibia implant to be adjusted to a level condition when bone cement is applied to the top surface of the cut tibia and the artificial tibia implant is driven and fixed into the top end of the cut tibia.

2. Description of the Related Art

As is well known, artificial knee joint substitution surgery, in which the knee joint that is damaged or deformed by congenital malformation, trauma, disease or degenerative arthritis, is widely performed.

Such an artificial knee joint substitution surgery is briefly described. After the bottom end of femur F and the top end of tibia T are cut respectively to replace the damaged knee joint with an artificial knee joint, bone cement is applied to the artificial femur implant 11 shown in FIG. 1 to attach it to the bottom end of the cut femur F. Next, after the artificial tibia implant 12 is driven into the top end of the cut tibia T, it is fixed with bone cement. At this time, in order to make relative motion smooth by minimizing the contact friction between the artificial femur implant 11 and the artificial tibia implant 12, an artificial polymer implant 13 as a buffer member is inserted and mounted on the top surface of the artificial tibia implant 12 fixed at the top end of the tibia. The artificial femur implant 11 and artificial tibia implant 12 inserted through such an operation process play the role of artificial knee joint 10 as a whole.

Here, the method of mounting the artificial tibia implant on the top end of tibia is described in more detail. First, a lengthwise drive-in slot H is formed so that the artificial tibia implant 12 is driven and fixed into the top surface of the cut tibia T. Next, after bone cement is applied to the top surface of the cut tibia T, the portion of the central axis A formed below the artificial tibia implant 12 is combined into the drive-in slot H of the tibia T, for the artificial tibia implant 12 to be adjusted to be level with the top surface of the tibia T. Then, bone cement is hardened to fix the tibia T and the artificial tibia implant 12 firmly in place. At this time, the artificial tibia implant 12 is adjusted so as to be in a level condition on the top surface of the cut tibia T through use of a tibia impacter. Namely, the tibia impacter applies impact from above the artificial tibia implant 12 to adjust the level position of the artificial tibia implant 12.

FIG. 2 illustrates one example of a conventional tibia impacter. Referring to this drawing, a conventional tibia impacter 1 is provided with a body 3, on which is vertically formed a handle portion 5 for the user to grip, and a striking portion 7 is monolithically formed at the top end of the body 3, and a pressing portion 9 is formed at the bottom end of the body 3. Accordingly, if the striking portion 7 is struck when the handle portion 5 is gripped, the pressing portion 9 presses the artificial tibia implant 12. The pressing force at this time hardens bone cement B while the artificial tibia implant 12 is adjusted so as to be in a level condition on the top surface of the cut tibia T. Here, the handle portion 5 and striking portion 7 are made of metallic material, and the pressing portion 9 is made of synthetic resin material so as to absorb impact. And the pressing portion 9 is composed of one pair of pressing structures with bilateral symmetry.

However, the conventional tibia impacter 1 composed like above has a problem that it is difficult to maintain the level condition accurately because the level condition is attained by adjusting the striking strength while confirming the thickness of bone cement with the naked eye only. Another problem is that because the central axis A of the artificial tibia implant 12 (i.e., the mechanical axis of tibia T) and the striking axis of the tibia impacter 1 are not on the same axis line but are eccentric, the artificial tibia implant 12 is installed inclined forward or backward even by a slight difference of striking force, as shown in FIG. 3.

### SUMMARY OF THE INVENTION

Accordingly, it is an object of the present invention to provide a tibia impacter having an alignable function by making the striking axis of tibia impacter coincide with the central axis of the artificial tibia implant and by vertically installing on one side of the tibia impacter a guide bar that helps to attain a level position of the artificial tibia implant through the mechanical axis of the tibia.

In accordance with the present invention, there is provided a tibia impacter having a body, a striking portion formed at the top end of the body, and a pressing portion formed at the bottom end of the body, which presses an artificial tibia implant through the pressing portion when the striking portion is pressed, so as to help the artificial tibia implant be mounted on the top surface of the cut tibia, the tibia impacter comprising: a guide bar which is installed vertically on one side of the tibia impacter for comparison with the mechanical axis of the tibia to compare the mechanical axis of the tibia and the guide bar, so that the level condition of the artificial tibia implant mounted on the top surface of tibia can be confirmed.

Preferably, the body of said tibia impacter is provided on one side with a protruded portion with an insert hole bored therethrough, and said guide bar is inserted vertically in said insert hole to maintain a fixed condition.

Preferably, the pressing portion of said tibia impacter is provided on one side with a protruded portion with an insert hole bored therethrough, and said guide bar is inserted vertically in said insert hole to maintain a fixed condition.

### BRIEF DESCRIPTION OF THE DRAWINGS

These and other objects, features, and advantages of preferred embodiments of the present invention will be more fully described in the following detailed description, taken in conjunction with the accompanying drawings. In the drawings:

FIG. 1 is a perspective view showing an artificial knee joint mounted instead of a damaged knee joint;

FIG. 2 is an exploded perspective view showing the configuration of a conventional tibia impacter;

FIG. 3 is a lateral view showing the method of mounting an artificial tibia implant on the top surface of tibia by using the conventional tibia impacter;

FIG. 4 is an exploded perspective view showing the configuration of a tibia impacter according to the present invention;

FIG. 5 and FIG. 6 are a lateral view and a front view showing the method of mounting an artificial tibia implant on the top surface of tibia by using the tibia impacter according to the present invention, respectively;

FIG. 7 and FIG. 8 are an exploded perspective view and a lateral view showing a tibia impacter according to another embodiment of the present invention, respectively.

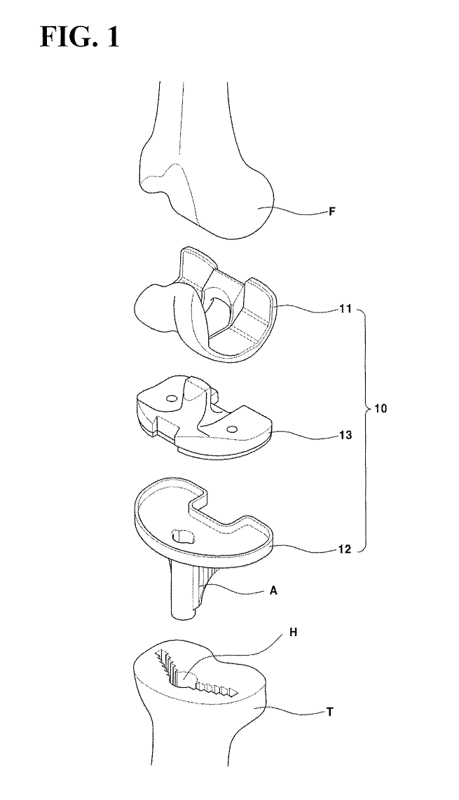

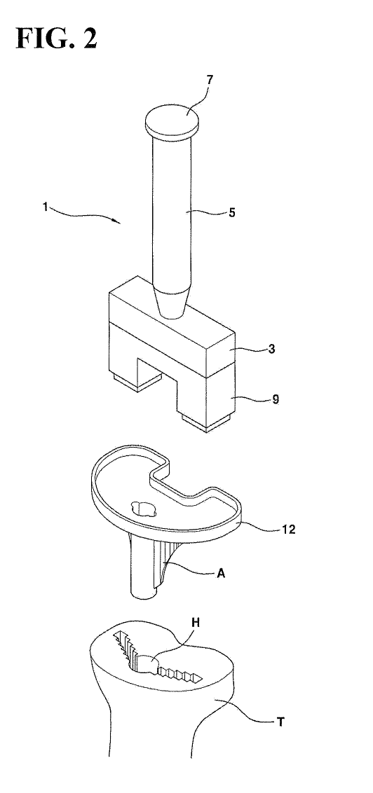

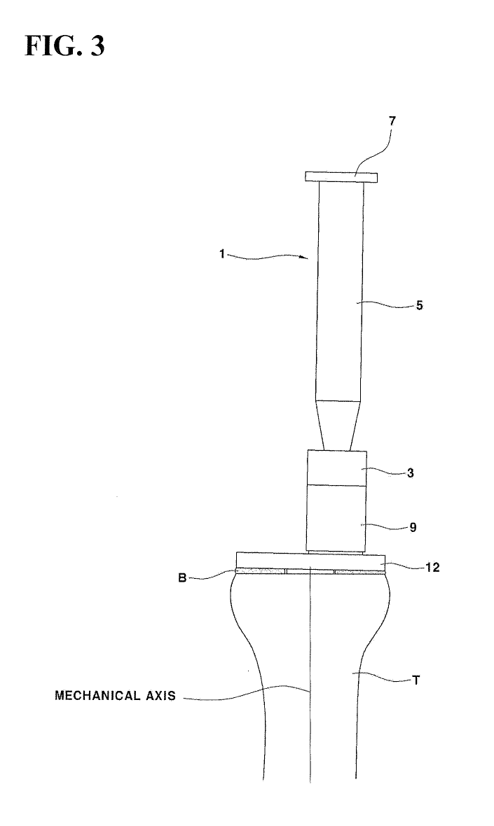

### DETAILED DESCRIPTION OF THE INVENTION

Below will be described in detail the present invention with reference to the accompanying drawings. And in the description below, the elements that are identical with those of the conventional structure are shown with identical symbols and duplicated description is omitted for simplification.

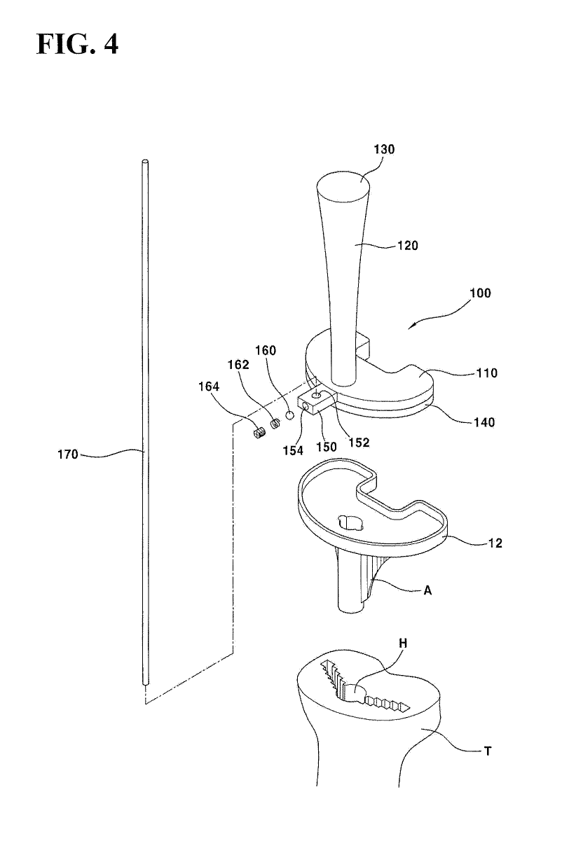

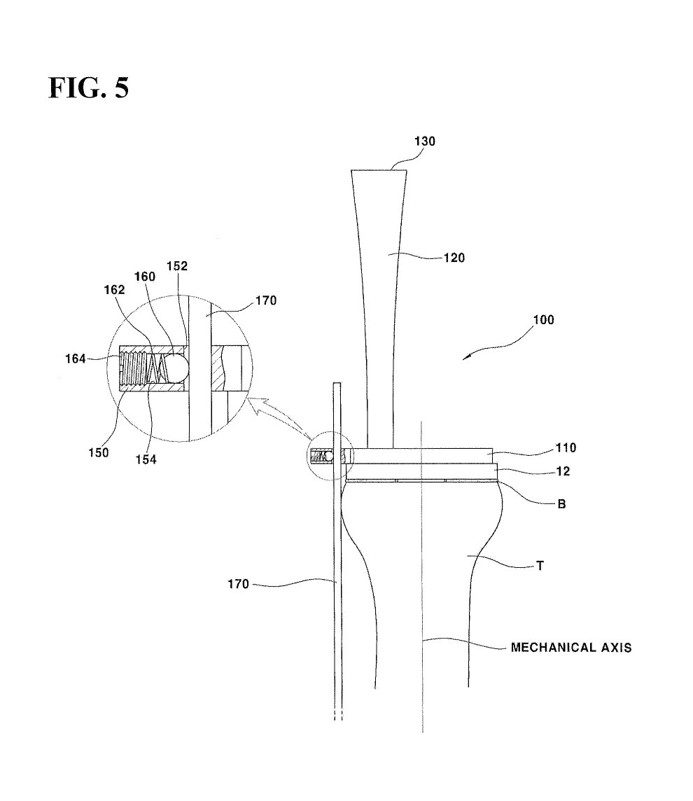

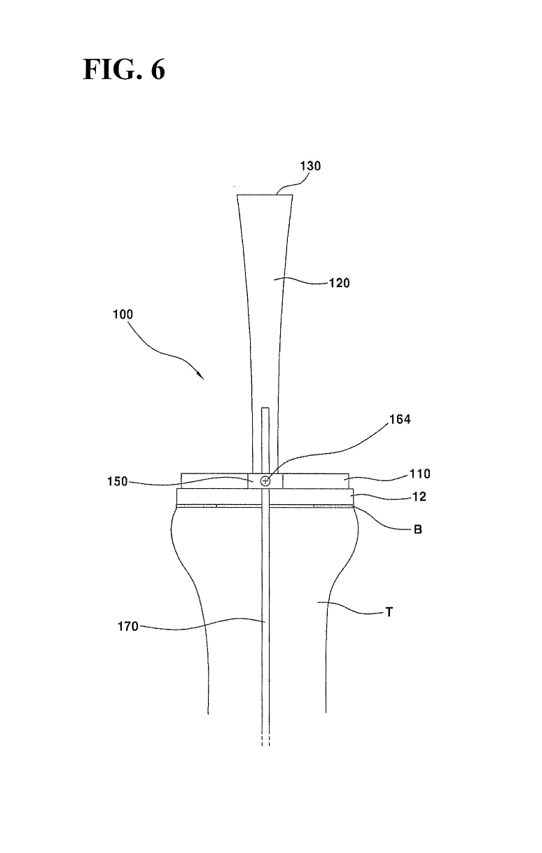

Referring to FIG. 4 to FIG. 6, a tibia impacter 100 according to the present invention is provided with a body 110, on the top surface of which is formed a vertically extended handle portion 120, wherein at the top end of the handle portion 120 is monolithically formed a striking portion 130, and at the bottom end of the body 110 is monolithically formed a pressing portion 140. Here, it is preferable that the body 110, handle portion 120 and striking portion 130 are made of metal or aluminum material so as to maintain strength when impact is applied, and the pressing portion 140 is made of synthetic resin material so as not to damage the surface of the artificial tibia implant 12. And it is preferable that the pressing portion 140 is made to have a shape identical with the artificial tibia implant so that uniform pressing force can be applied on the whole, and that the striking portion 130 is made to be positioned on an axis line identical with the central axis A of the artificial tibia implant 12 so that the striking point is in an accurate position without being eccentric from the central axis A of the artificial tibia implant 12.

Next, a guide bar 170 is installed vertically on one side of the tibia impacter body 110 to be compared with the mechanical axis of tibia T so as to compare the mechanical axis of tibia T and the vertical line of the guide bar 170, so it is possible to confirm the level condition of the artificial tibia implant 12 mounted on the top surface of tibia T. Namely, if the mechanical axis of the tibia T and the vertical line of the guide bar 170 coincide, the artificial tibia implant 12 is maintaining a level condition; if they do not coincide but are deviated, the artificial tibia implant 12 is installed inclined as much as the deviation.

Here, for installing the guide bar 170, the present invention provides a structure that a protruded portion 150 with an insert hole 152 bored therethrough is formed on one side of the tibia impacter body 110, and the guide bar 170 is driven into this insert hole 152 so as to maintain a fixed condition. At this time, the usual check ball mechanism is applied for the structure of fixing the guide bar 170. Namely, an assembly hole 154 is formed on one side of the protruded portion 150, and a fixing ball 160 and spring 162 are inserted into this assembly hole 154 and fixed with a bolt 164, this is such that the fixing ball 160 presses the guide bar 170 to make it immovable. Of course, the present embodiment illustrates the check ball mechanism as a method for making the guide bar 170 immovable, but as long as the guide bar 170 can be fixed at a desired position by inserting it into the insert hole 152, any method may be applied, whether it is a pin method or a hook method.

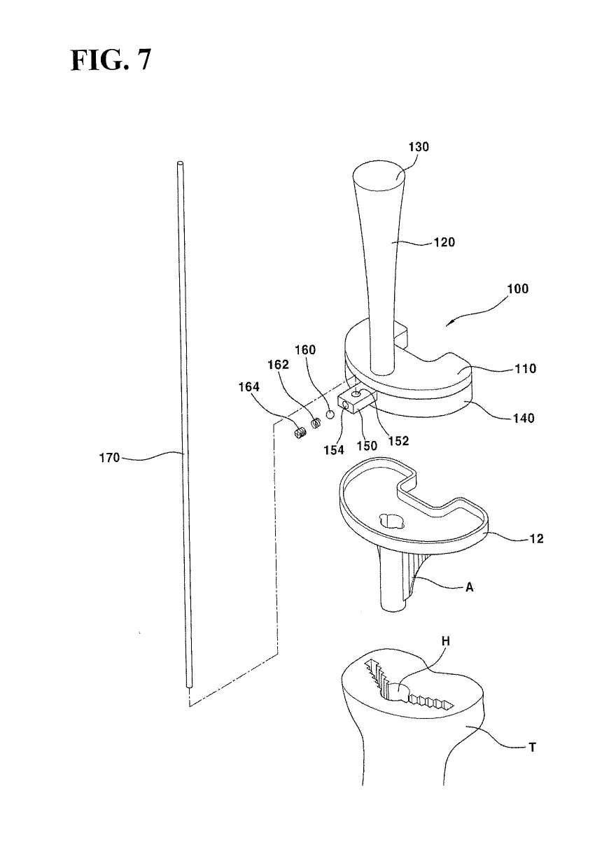

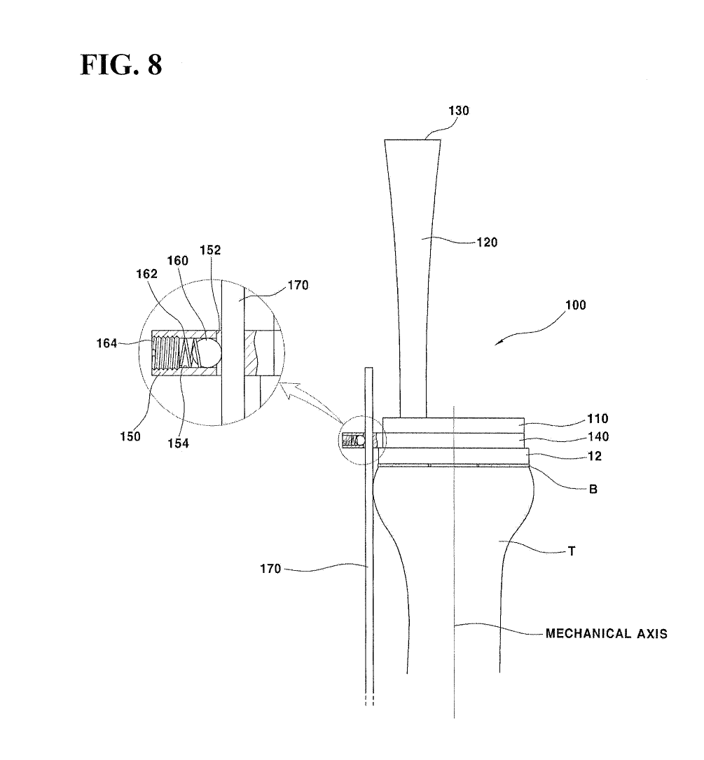

Meanwhile, in the embodiment described above, a protruded portion 150 is formed on one side of the body 110 to fix the guide bar 170, but as shown in FIG. 7 and FIG. 8, it may also be configured in such a way that a protruded portion 150 with an insert hole 152 bored therethrough is formed on one side of the pressing portion 140 and a guide bar 170 is fixed in the protruded portion 140. Namely, the present invention is characterized in that a guide bar 170 is vertically installed immovably on one side of the tibia impacter 100, but any method may be applied as long as the guide bar 170 can be installed immovably.

Below will be described the method of mounting an artificial tibia implant on the top surface of the cut tibia by using a tibia impacter according to the present invention configured like above, and the operation and effects according to that.

First, apply bone cement B on the top surface of the cut tibia T and then install an artificial tibia implant 12 in such a way that the central axis A of the artificial tibia implant 12 is inserted in the drive-in slot H formed on the tibia T. Next, lay the tibia impacter 100 according to the present invention on the top surface of the artificial tibia implant 12, and strike the striking portion 130 of the tibia impacter 100 so that the pressing portion 140 presses the artificial tibia implant 12 so as to come into close contact with the top surface of the tibia T.

At this time, while confirming whether the vertical line of the guide bar 170 and the mechanical axis of the tibia T are on the same vertical axis line, press the artificial tibia implant 12. Namely, if the mechanical axis of the tibia T and the vertical line of the guide bar 170 coincide, the artificial tibia implant 12 is maintaining a level condition; if they do not coincide but are deviated, the artificial tibia implant 12 is installed inclined as much as the deviation.

Therefore, it is possible to change the striking position as much as it is inclined to keep a level condition. Accordingly, the present invention is provided with an alignment guide function, so the artificial tibia implant 12 can be installed in the level condition easily.

According to the present invention, the guide bar is installed vertically on one side of the tibia impacter to make it possible to easily confirm the level condition of the artificial tibia implant installed on the top surface of the tibia while comparing the mechanical axis of tibia and the vertical line of the guide bar, so it is possible to easily install an artificial tibia implant accurately without an error. This has effects that the operation time is shortened, medical accidents are decreased remarkably, and medical stress is reduced.

Although the present invention has been described in detail reference to its presently preferred embodiment, it will be understood by those skilled in the art that various modifications and equivalents can be made without departing from the spirit and scope of the present invention, as set forth in the appended claims.

What is claimed is:

1. A tibia impacter having a body, a striking portion formed at the top end of the body, and a pressing portion formed at the bottom end of the body, which presses an artificial tibia implant through the pressing portion when the striking portion is pressed, so as to help the artificial tibia implant be mounted on the top surface of the cut tibia, the tibia impacter comprising:
a guide bar which is installed vertically on one side of the tibia impacter for comparison with the mechanical axis of the tibia to compare the mechanical axis of the tibia and the guide bar, so that the level condition of the artificial tibia implant mounted on the top surface of tibia can be confirmed.

2. The tibia impacter of claim 1, wherein the body of said tibia impacter is provided on one side with a protruded portion with an insert hole bored therethrough, and said guide bar is inserted vertically in said insert hole to maintain a fixed condition.

3. The tibia impacter of claim 1, wherein the pressing portion of said tibia impacter is provided on one side with a protruded portion with an insert hole bored therethrough, and said guide bar is inserted vertically in said insert hole to maintain a fixed condition.
